(19) United States
(12) Patent Application Publication
(10) Pub. No.: US 2018/0187124 A1
(43) Pub. Date: Jul. 5, 2018

(54) VEGETABLE COMPOSITION TO BE USED FOR COATING GRANULATE PRODUCTS

(71) Applicant: Bejos SpA, Antofagasta (CL)

(72) Inventor: Mauricio Daniel SVRIZ VIALE, Antofagasta (CL)

(21) Appl. No.: 15/855,621

(22) Filed: Dec. 27, 2017

Related U.S. Application Data
(60) Provisional application No. 62/442,232, filed on Jan. 4, 2017.

(51) Int. Cl. C11C 1/00; C11B 3/16; C07C 53/126; C07C 57/03; C09D 191/00

(52) U.S. Cl. CPC C11C 1/002; C11B 3/16; C09D 191/00; C07C 57/03; C07C 53/126

(57) ABSTRACT

A mixture of vegetable oils having different predominance in fatty acid chains, such as palmitic acid, oleic acid and linoleic acid is provided. This vegetable composition is added to granulated products to improve their physical properties, such as hardness, abrasion resistance, impact resistance and hygroscopicity. When transported in large volume, these products tend to wear out generating losses of material which affect both producers and buyers. Thanks to the properties provided by the present invention, the granulated products manage to preserve their physical properties, reducing their degradation. Finally, a method for adding the vegetable composition to granulated products is disclosed.

### CROSS REFERENCE TO RELATED APPLICATION

This application claims the benefit of priority of U.S. Provisional Patent Application No. 62/442,232, filed on Jan. 4, 2017, which is incorporated herein in its entirety.

### FIELD OF THE INVENTION

The present invention relates to a composition comprising a mixture of vegetable oils having different predominance of fatty acid chains, such as palmitic acid, oleic acid and linoleic acid. This vegetable composition is added to granulated products to improve their physical properties, such as hardness, abrasion resistance, impact resistance and hygroscopicity. When transported in large volume, these products tend to wear out generating losses of material which affect both producers and buyers. Thanks to the properties provided by the present invention, the granulated products manage to preserve their physical properties, reducing their degradation.

### STATE OF THE ART

Granulated products must retain their physical properties in order to be effective when used, and also avoid generating pollution during loading, unloading and transport process.

In some cases, granulated products absorb moisture generating agglomeration with each other, increasing their size, or altering their physical properties. In the same way, these granulated products are regularly affected by the impact and friction generated by the movement of the particles during transport, loading and unloading. This causes a problem when they are used in bulk, since their physical properties are altered, particularly their morphology and size, further generating high pollution, which complicates bulk handling mostly in ports or areas of application.

Prior art U.S. Pat. No. 9,346,976 B2 provides an industrial fluid made from non-hazardous, biodegradable materials, the use of which allows to avoid or substantially reduce instances of mining material sticking or freezing in mining and mineral processing equipment. Said invention discloses an industrial fluid comprising a natural oil (such as crude skimmed soybean oil) and a surfactant compound.

The present application seeks to avoid the loss of material during product transfer, also facilitating the bulk handling. This is achieved by applying a composition comprising a mixture of different vegetable oils, such as sunflower, soybean, corn, palm, coconut, palm seed, peanut oil, among other, which have different predominance in fatty acid chains.

Unlike what is proposed in U.S. Pat. No. 9,346,976 B2, the vegetable composition of the present invention does not use surfactants within its composition and likewise manages to avoid or diminish instances of adhesion or freezing. In addition, the vegetable composition herein allows to work with the bulk product and at a gradual dissolution for its final application. Additionally, there is the possibility of preparing a vegetable composition with a particular coloring to meet the requirements of some countries on fertilizer pigmentation, requested by some users.

### Solution to the Technical Problem

Various methods have been evaluated in the area of non-metallic mining to protect granulated products (pellets), because the friction generated between the particles during their transfer produces a pulverized material that makes difficult use thereof and undermines their bulk commercialization, further limiting their use in this format.

The composition of the present invention solves said state of the art problem, since it provides the following comparative advantages:
a) The product coated with the vegetable composition described in the present invention has a lower loss due to abrasion and impact, a higher hardness and a lower moisture adsorption; properties that together improve the conditions of transport and bulk handling thereof. In the particular instance of the agricultural sector, the use of the vegetable composition described herein as a fertilizer additive allows to reduce the pollution and to achieve a gradual solubilization of the granulated product when applied, maintaining the size and porosity of the original grain.
b) The product has a lower cost per processed ton, due to the lower dosage required compared to the use of other additives. The product described in the present invention needs a dosage between 1 to 4 kg of vegetable composition per ton of granulated product to be coated.
c) The vegetable composition described in the present invention corresponds to a biodegradable product, because it can be decomposed by biological agents releasing the main elements thereof (carbon, hydrogen and oxygen).

### DESCRIPTION OF THE FIGURES

FIG. 1: Shows a graph of abrasion analysis.

FIG. 2: Shows a graph of hardness analysis.

FIG. 3: Shows a graph moisture analysis.

### DESCRIPTION OF THE INVENTION

The present invention discloses a composition from a plant source comprising a mixture of vegetable oils intended for the coating of various types of products in pellet or granulated form to avoid altering the chemical properties thereof, and thus not affecting their effectiveness and/or functionality when transported.

The composition is generated by mixture of two or more oils having high levels of oleic or linoleic acid (unsaturated fatty acids) on the one hand, and oils with high levels of palmitic, myristic or lauric acid (saturated fatty acids) on the other hand.

The fatty acid composition of the present invention comprises a content in the range from above 0 to 99% oleic acid, a range from above 0 to 99% linoleic acid, a range from above 0 to 99% palmitic acid, a range from 0 to 99% myristic acid, a range from 0 to 99% lauric acid, and may also contain other fatty acids having a 4 to 24 carbon chain, such as, for example, caproic acid, caprylic acid, capric acid, myristoleic acid, palmitoleic acid, heptadecanoic acid, stearic acid, linolenic acid, arachidic acid, among others.

In a preferred invention, the fatty acid composition of the product has a content from 1 to 50% oleic acid, 1 to 60% linoleic acid, 1 to 55% palmitic acid, 0 to 10% myristic acid and 0 to 10% lauric acid, and may also contain other fatty acids having a 4 to 24 carbon chain, such as, for example, caproic acid, caprylic acid, capric acid, myristoleic acid, palmitoleic acid, heptadecanoic acid, stearic acid, linolenic acid, arachidic acid, among others.

In another preferred invention, the fatty acid composition of the product has a content from 10 to 35% oleic acid, 30 to 60% linoleic acid, 1 to 25% palmitic acid, 0 to 8% myristic acid and 0 to 8% lauric acid and may also contain other fatty acids having a 4 to 24 carbon chain, such as, for example, caproic acid, caprylic acid, capric acid, myristoleic acid, palmitoleic acid, heptadecanoic acid, stearic acid, linolenic acid, arachidic acid, among others.

In another preferred invention, the fatty acid composition of the product has a content from 1 to 50% oleic acid, 1 to 25% linoleic acid, 1 to 50% palmitic acid, 0 to 30% myristic acid and 0 to 60% lauric acid, and may also contain other fatty acids having a 4 to 24 carbon chain, such as, for example, caproic acid, caprylic acid, capric acid, myristoleic acid, palmitoleic acid, heptadecanoic acid, stearic acid, linolenic acid, arachidic acid, among others.

In another preferred invention, the fatty acid composition of the product has a content from 5 to 30% oleic acid, 1 to 60% linoleic acid, 1 to 20% palmitic acid, 0 to 25% myristic acid and 0 to 60% lauric acid, and may also contain other fatty acids having a 4 to 24 carbon chain, such as, for example, caproic acid, caprylic acid, capric acid, myristoleic acid, palmitoleic acid, heptadecanoic acid, stearic acid, acid linolenic, arachidic acid, among others.

In addition, the invention discloses a method for obtaining the vegetable composition of the present invention comprising the following steps:
a) Heating vegetable oils having a higher percentage of unsaturated fatty acids between 20° C. and 50° C.
b) Heating vegetable oils having a higher percentage of saturated fatty acids between 30° C. and 80° C.
c) Mixing both fractions prepared as described in steps a) and b);
d) Mechanically stirring the mixture of stage c) at a speed from 10 to 500 rpm, from 5 to 60 minutes, reducing the temperature until reaching room temperature, to obtain a homogeneous mixture and without the presence of lumps due to oils with higher percentage of saturated fatty acids.

The conditions for preparing the vegetable composition of the present invention are diverse and depend on the properties sought to be strengthened in the pellet, promoting the mixture of oils high in lauric, myristic, palmitic, oleic and/or linoleic acids. For vegetable oils having a higher concentration of lauric, myristic or palmitic acids (saturated fatty acids), a heating step is required for their melt because they are in solid state at room temperature, while vegetable oils high in oleic or linoleic acids (unsaturated fatty acids) are generally found in liquid state at room temperature. When any of the oils to be mixed is in solid state, it must be heated until it is liquid and then mixed. The mixing ratios will define compound characteristics, and therefore the properties to be improved when dosing to the pellet.

The vegetable composition described in the present invention allows the addition of micronized iron oxides to provide the pellet with a red coloration, in accordance with the identification standards of granulated products for fertilizer purposes in certain countries. The pellet maintains a high degree of dispersion for its subsequent spraying, generating a reddish film with code number 120 or 130 (industrial code) on the outer part of the pellet, while inside remains a white color. This pigment allows the pellets being eventually fractionated to re-stain with the coloring by friction with other pellets.

The vegetable composition of the present invention obtained by the method described above is added to the granulated product by spraying or dripping the vegetable composition onto the product, which is mechanically stirred in a horizontal homogenizer from 5 to 60 minutes. The dosage of vegetable composition added to the granulated product should be from 1 to 4 kg of vegetable composition per ton of granulated product. The methodology for adding the vegetable composition to the granulated product is not exclusively limited to the forms described in the present invention.

### APPLICATION EXAMPLES

Four assays were carried out for coating the granulated product of potassium chloride (KCl), which is used industrially as a fertilizer. For this purpose, 4 mixtures were prepared using the method described herein.

### Example 1

For the first vegetable composition (Mixture A), two vegetable oils, crude soybean oil and palm oil were used in a ratio of 50%-50% by volume. These oils were heated at 50° C. to melt the palm oil. They were then mixed under stirring at 100 rpm for 10 minutes. The mixture resulted in a composition of 0.1% lauric acid, 0.5% myristic acid, 27.0% palmitic acid, 31.2% oleic acid and 30.6% linoleic acid. The vegetable composition was then added to KCl by spraying, while the granulated product (KCl) was mixed in a horizontal homogenizer for 10 minutes. The dosage used was 3 kg of vegetable composition/ton of product.

### Example 2

For the second vegetable composition (Mixture B), two vegetable oils, crude soybean oil and palm oil, were used in a ratio of 93%-7% by volume. These oils were heated at 50° C. to melt the palm oil. They were then mixed under stirring at 100 rpm for 10 minutes. The mixture resulted in a composition of 0% lauric acid, 0.1% myristic acid, 12.8% palmitic acid, 23.8% oleic acid and 48.4% linoleic acid. The vegetable composition was then added to KCl by spraying, while the granulated product (KCl) was mixed in a horizontal homogenizer for 10 minutes. The dosage used was 3 kg of vegetable composition/ton of product.

### Example 3

For the third vegetable composition (Mixture C), two vegetable oils, palm oil and palm kernel oil, were used in a ratio of 50%-50% by volume. These oils were heated at 55° C. to melt both palm oil and palm kernel oil. They were then mixed under stirring at 100 rpm for 20 minutes. The mixture resulted in a composition of 24.1% lauric acid, 8.9% myristic acid, 26.0% palmitic acid, 27.3% oleic acid and 8.3% linoleic acid. The vegetable composition was then added to KCl by spraying, while the granulated product (KCl) was mixed in a horizontal homogenizer for 10 minutes. The dosage used was 2 kg of oil/ton of product.

### Example 4

For the fourth vegetable composition (Mixture D), two vegetable oils, corn oil and palm kernel oil, were used in a ratio of 93%-7% by volume. These oils were heated at 50° C. to melt the palm kernel oil. They were then mixed under stirring at 100 rpm for 10 minutes. The mixture resulted in a composition of 3.7% lauric acid, 1.2% myristic acid, 10.4% palmitic acid, 26.4% oleic acid and 49.9% linoleic acid. The vegetable composition was then added to the KCl by spraying, while the granulated product (KCl) was mixed in a horizontal homogenizer for 10 minutes. The dosage used was 3 kg of oil/ton of product.

### Results

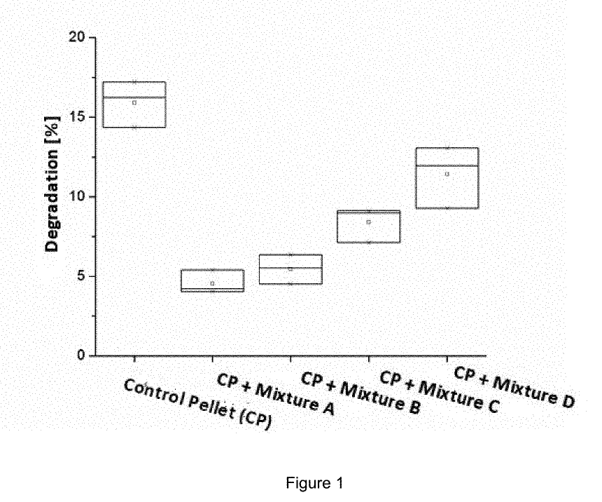

FIG. 1 shows the effect in the granulated product abrasion level of adding the vegetable composition onto the fertilizer pellets. The pellets were coated with 4 different vegetable compositions. Subsequently, samples were subjected to a mechanical abrasion process. To carry on this process, 50 grams of pellets were added to a number 8 sieve (2.36 mm), together with 10 stainless steel balls, and subjected to agitation on a sieve shaker, for 10 minutes. The abrasion level was quantified as the percentage of husked pellets, which corresponds to the passing fraction of the sieve. FIG. 1 shows that mixtures of vegetable oils decreased pellet degradation between 5 to 10%.

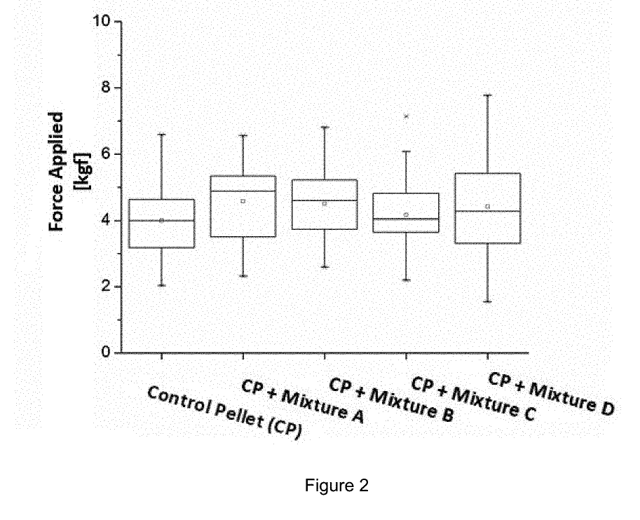

FIG. 2 shows the effect in the granulated product hardness level of adding the vegetable composition onto the fertilizer pellets. The pellets were coated with 4 different vegetable compositions. Subsequently, the samples were subjected to a vertical force, quantifying the force required to fracture them. This study was carried out with a quantity of 35 selected pellets. In commercial preparations, the pellets must be able to resist 3 kgf of applied force before fracturing. This figure shows that the coating disclosed in the present invention does not affect the hardness level of the pellet.

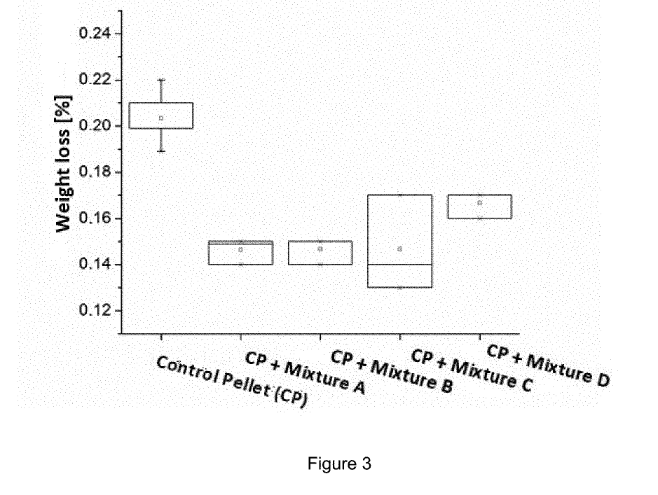

FIG. 3 shows effect in the granulated product moisture level of adding the vegetable composition onto the fertilizer pellets. The pellets were coated with 4 different vegetable compositions. Subsequently, the retained moisture of each sample was quantified applying heat. For this purpose, 10 grams of pellets were added in a thermobalance, and the loss of weight was recorded by subjecting the sample to 105° C. for 20 minutes. The figure shows that addition of a vegetable composition decreases loss of weight by heat, indicating that the coated pellet is less likely to absorb moisture relative to the control sample (pellet without additives).

Therefore, FIGS. 1, 2 and 3 show the physical parameters of the coated pellet versus the uncoated pellet (control). The abrasion analysis (sheet 1/3) showed that the pellets coated with the vegetable composition described herein reduced from 5% (mixture D) to 12% (mixture A) the degradation produced by the treatment, in comparison with the uncoated pellet. The hardness analysis (sheet 2/3) showed that all the pellets coated with the vegetable composition described herein required at least 4 kgf to be fractured, being more resistant relative to the uncoated pellet. Moisture analysis (sheet 3/3) of the pellets coated with the vegetable composition described herein losses less weight by heat treatment than uncoated pellets, whereby the coating allowed the pellet to absorb less environmental moisture.

1. Vegetable composition for adding to granulated products to improve and/or maintain physical properties thereof when transported, comprising:
a) an oleic acid range from over 0 to 99%;
b) a linoleic acid range from over 0 to 99%;
c) a palmitic acid range from over 0 to 99%.

2. Vegetable composition according to claim 1, comprising:
a) an oleic acid range from over 1 to 50%;
b) a linoleic acid range from over 1 to 60%;
c) a palmitic acid range from over 1 to 55%.

3. Vegetable composition according to claim 2, comprising:
a) an oleic acid range from 10 to 35%;
b) a linoleic acid range from 30 to 60%;
c) a palmitic acid range from 1 to 25%.

4. Vegetable composition according to claim 2, comprising:
a) an oleic acid range from 1 to 50%;
b) a linoleic acid range from 1 to 25%;
c) a palmitic acid range from 1 to 50%.

5. Vegetable composition according to claim 3, comprising:
a) an oleic acid range from 5 to 30%;
b) a linoleic acid range from 1 to 60%;
c) a palmitic acid range from 1 to 20%.

6. Vegetable composition according to claim 1, further comprising a myristic acid range from 0 to 99% and/or a lauric acid range from 0 to 99%.

7. Vegetable composition according to claim 6, wherein the myristic acid range is from 0 a 30% and/or lauric acid range is from 0 a 60%.

8. Vegetable composition according to claim 7, wherein the myristic acid range is from 0 a 25% and/or lauric acid range is from 0 a 60%.

9. Vegetable composition according to claim 8, wherein the myristic acid range is from 0 a 10% and/or lauric acid range is from 0 a 10%.

10. Vegetable composition according to claim 9, wherein the myristic acid range is from 0 a 8% and/or lauric acid range is from 0 a 8%.

11. Vegetable composition according to claim 1, further comprising other fatty acids having a 4 to 24 carbon chain selected from caproic acid, caprylic acid, capric acid, myristoleic acid, palmitoleic acid, heptadecanoic acid, stearic acid, linolenic acid, arachidic acid, among others.

12. Vegetable composition according to claim 1, further comprising a pigment.

13. Method for obtaining the vegetable composition according to claim 1, comprising the following steps:
a) heating vegetable oils having a higher concentration of oleic and linoleic acids (unsaturated fatty acids) between 20° C. and 50° C.;
b) heating vegetable oils having a higher concentration of lauric, myristic or palmitic acids (saturated fatty acids) between 30° C. and 80° C.;
c) mixing both fractions obtained from of steps a) and b); and
d) mechanically stirring a mixture obtained from stage c) at a speed of from 10 to 500 rpm, from 5 to 60 minutes, reducing the temperature until reaching from 20 to 25° C. to obtain a homogeneous mixture.

14. Method for adding the composition of claim 1 to granular products, wherein the vegetable composition is added by spraying or dripping to the granulated product, which is agitated mechanically.

15. Method for adding the composition according to claim 14 to granular products, wherein the dosage of the vegetable composition added to the granulated product should be from 1 to 4 kg of vegetable composition per ton of granulated product.
